(19) United States
(12) Patent Application Publication
(10) Pub. No.: US 2008/0026377 A1
(43) Pub. Date: Jan. 31, 2008

(54) Skin wrinkle application of genomic replacement therapy

(76) Inventors: Nicholas Anthony Laszlo, Knoxville, TN (US); John M. Pavelchik, Knoxville, TN (US)

Correspondence Address: John M. Pavelchik, 2827 White Oak Lane, Knoxville, TN, 37917, US

(21) Appl. No.: 11/494,200

(22) Filed: Jul. 28, 2006

(51) Int. Cl. A61K 48/00; C12Q 1/68

(52) U.S. Cl. 435/6; 514/44

(57) ABSTRACT

Genomic Replacement Therapy uses the human genome found in mantle dentin of a patient's tooth to affect other cells of the body. Mantle Dentin cells in the developed tooth do not reproduce, therefore the DNA is “younger”. Replacing the genome with a “younger” one will hopefully return youthful appearances to clients.

### CROSS REFERENCE TO RELATED APPLICATIONS

### U.S. Patent Documents

U.S. Pat. No. 5,885,829 March 1999 Mooney et all

### STATEMENT REGARDING FEDERALLY SPONSORED RESEARCH OR DEVELOPMENT

Not applicable

### REFERENCE TO SEQUENCE LISTING, A TABLE, OR A COMPUTER PROGRAM LISTING COMPACT DISC APPENDIX

Not applicable

### BACKGROUND OF THE INVENTION

This invention lies in the field of medical or biological science. The range of the application will include cosmetic and medical functions. The application will include a treatment the restoration of proteins such as collagen and elastin. The procedure is similar to both mammalian cloning and to gene replacement therapy.

In mammalian cloning, an egg with the original genetic material removed is injected with genetic material from another mammal. This was performed by Dr. Ian Wilmut's team and resulted in the first cloned mammal, “Dolly” the sheep. The techniques here include the removal of a full genome and placing it in an egg. In gene replacement therapy, a damaged or harmful gene is replaced with a copy created in the laboratory. If the gene is accepted into the cell, then the phenotypic result should become evident as the patient's cells express themselves and/or reproduce. This technique includes the identification of the gene of interest, the creation of the replacement gene, it's incorporation into a vector, and the use of the vector to introduce the new material. In the scope of this patent, we will be using the extraction of the genome, the incorporation of the genome into a vector, and it's insertion into a diploid cell.

Currently, there are a number of different skin care treatments that are designed to reduce wrinkles and restore collagen and elastin levels. These must be reapplied, as the effects are designed to cover the phenotypic effect caused by the aging genotype. There is a concentration on fixing proteins and polypeptides that are eroded by the effects of aging, such as the introduction of collagen, but there is no mainstream concentration on the regeneration of the genes that are creating the effect. The cosmetic industry is a billion dollar business, which is designed to enhance a person's looks with artificial colors, smooth out a persons natural features, or reduce the look of aging.

The market for anti-aging products currently carries many approaches to the problem. One commonly used technique is to inject dead Botulism Type A into the muscles around the face to reduce wrinkles (4). Another uses skin firming agents, elasticizers, and skin hydrators to make the skin look smoother and firmer (5). This requires repeated applications, several times a day, to be effective. More extreme measures, such as surgical manipulation, have been used for some time to reduce the effects of larger wrinkles and sagging skin. These procedures can require a hospital stay, and pain medication. For the face, these usually involve cutting the skin in an inconspicuous place, pulling on the skin to remove the most prominent wrinkles, and surgically reattaching the skin. One of the undesired effects is that the natural openings in the skin, such as the mouth and eyes, can also become stretched and can thus appear malformed, especially when compared to the rest of the face. The procedure will usually require recovery time for the patient, and there is no guarantee that the surgery will not need to be performed again. The process is also non-reversible.

As any living thing ages, the DNA becomes less like it was at birth. This is due to carcinogens, oxidative reduction reactions, and to random mutations that are not repaired, or are done so incorrectly, by our cells. (11) In humans, this can lead to skin collagen and elastin loss. All of these phenotypic responses are caused from the genes controlling them being affected by simple aging.

In the permanent teeth of a human, the DNA stops replicating once the tooth is fully developed. The process of developing these teeth begins at around age six. The tooth of a human grows by allowing new material to develop from the center, and push the old material towards the sides. This will mean that the youngest genetic material will be seen at the edge just below the enamel, in an area called the mantle dentin. Whatever genetic alterations happen to the body will usually occur to reproducing cells. (13) As the fully developed permanent teeth have none, the DNA is unaffected. A person whom is eighty years old will still have six-year-old genetics within them, if the teeth are still present. We are suggesting that the genome found here can be used to fix the damage caused later in life in other cells.

### BRIEF SUMMARY OF THE INVENTION

The aforementioned problems have genetic links. The idea behind this invention is that the genome of affected cells can simply be replaced with DNA that a person already has within them. Once the genome is replaced, some genetic problems will no longer exist.

Inside the permanent tooth of a person, excluding the wisdom teeth that can develop later, there is a layer of mantle dentin. This layer of cells contains DNA that was created when the person was about 6 years old. Since the cells here do not reproduce, there will be no genetic difference in these cells from the moment they were created until their demise. Despite the rest of the body's constant cycling of cells, the teeth remain the same. This DNA can be extracted, introduced into a vector, and used to replace the genome of other cells in the body that have had genetic damage. If the genotype of one cell in the body is virtually the same as any other genome-bearing cell in the body, then the DNA is interchangeable. The cells simply express what is needed from the DNA, according to what the cell is and what the cell is supposed to do. If the DNA from the tooth is unchanged from it's creation at around six-years-old, then the cell it is introduced to will read from six-year-old DNA, despite the age of the person.

If age is viewed as a genetic problem, then genome replacement will have a key effect on skin properties and hair growth. Since hair loss, as well as hair pigment loss, is a genetic factor for most, the application can be used to regrow and recolor hair. As soon as the cell has the genetic blueprint for hair growth, the rest of the cell can perform to the task. Skin that has been exposed to tanning beds and/or natural light over several years can be exfoliated and introduced to the new genetic material. This should allow the skin to regrow collagen, elastin, and any other protein or polypeptide that it could at the age of six, and at the same levels. This can be performed anywhere on the skin, not just the face. This can be a great advantage from agents such as skin creams that are currently used primarily on the face and neck, as this more permanent solution can be used anywhere on the same person. The result can be more effective than current skin rejuvenation creams as well, as the entire genetic make-up will be reset to the point of a child, despite the person's age.

### BRIEF DESCRIPTION OF THE SEVERAL VIEWS OF THE DRAWING

Not applicable.

### DETAILED DESCRIPTION OF THE INVENTION

All working surfaces will be decontaminated with a wash sequence of sodium hypochlorite (10%), hydrochloric acid (0.1%), and ethanol (70%). All equipment will be sterilized by autoclaving and decontaminated by exposure to ultraviolet light and bleaching. Novocain will be used to numb the tooth at the gums. A cordless, variable-speed, hand-held electric drill with a 1.0-1.5 mm drill bit, will be used to obtain 0.01-0.02 g of mantle dentin powder from a tooth. A drilling speed of less than 100 revolutions per minute (r.p.m.) was used to minimize heat production, which could result in DNA degradation. The hole or holes drilled will be approximately 1.5-2.0 mm wide and 2.0-3.0 mm deep. Holes will be drilled preferably at the base of the gum line, or on the inside of the tooth to minimize visible damage. Prior to drilling, the drill site will be cleaned with 70-100% ethanol to remove dust and particulate matter. Cotton will be used to keep the area dry. A new, autoclaved drill bit and autoclaved collection tray made from aluminum foil will be used for each patient. The head will be held at an incline during drilling to ensure the tooth powder produced falls into the collection tray. The tooth will be filled as if it were any other dental hole. Tooth powder will be transferred from the tray to a sterile 2 mL tube by careful decanting. This will be packed in ice and stores in a thermal container to be sent to a lab, if the lab work will not be performed on site. After the drilling of each tooth, drill bits and all disposable equipment, including gloves, will be discarded, and working surfaces decontaminated, as described above. (14),(15)

The tooth powder sample is added to a lysis buffer containing alpha-casein. Next, guanidine thiocyanate (GuSCN) and silica are introduced for 10 minutes. After centrifuging, the supernatant will be removed, and the pellet will be washed with 1 ml of acetone, the process is repeated at least three times, until the sample remaining is pure DNA from the mantle dentin. (16)

PCR can be used to replicate the genome, as long as a specific primer is not used to isolate a single gene. This will require the DNA, a solution of primers to start the reaction, and a healthy supply of base pairs (Adenine, Guanine, Cytosine, and Thymine). PCR uses a strand of DNA, in this case one for each chromosome, and heats it to 96 degrees C. to separate the DNA strands from their hydrogen bonds. These are then lowered to 68 degrees C. to allow the primers to attach to the template strands of DNA. Once it is lowered to 72 degrees C., the new strands are allowed to recombine. This temperature will need to be maintained for about four hours, to allow for the entire genome to replicate. This will allow one strand to become two. The next cycle of these temperature changes will allow two strands to become four. After twenty cycles, over a million strands are present. This can be accomplished in three days. An alternative to PCR is to grow the mantle dentin from a small sample. This process will allow for more cells to be grown, thus more DNA. Once a sufficient supply of DNA is present, the solution can be introduced to a detergent to create a vector. This will create a Detergent-DNA complexes. One of the most common methods is to use a non-ionic detergent (e.g., lipofectin) that forms a complex with the DNA and by mechanisms still not well understood allow for introduction of DNA into the cell. (17) Some of the solution will be stored in a cold climate for preservation, to ensure that future application will not require more extraction from the patient. What is required for the current application will be placed in the necessary form and distributed.

If introduced as a cosmetic, the patient must first use an abrasive exfoliate, such as a pumice scrub, to relieve any dead skin from the area. Next, the solution will be placed into a hand/body cream that also contains propylene or butylene glycol, glycerine or glyceryl stearate, stearic acid or linoleic acid, sorbitan stearote, and urea, as do most body lotions. After it is applied and the cells accept the DNA, the skin and hair should revert back to the phenotypic properties seen at approximately six years of age.

### REFERENCES

(1), (2) http://www.bosley.com

(3) http://www.hairsite.com/color/color-technical.htm

(4) http://www.botox.com

(5) http://www.strivectin-kleinbecker.comnFAQ.php?question=FAQ1. Txt

(6) D L Tait, P S Obenmiller, S Redlin-Frazier, R A Jensen, P Welcsh, J Dana, M C King, D H Johnson, and J T Holt, A phase I trial of retroviral BRCA1sv gene therapy in ovarian cancer Clin. Cancer Res. 1997

(7) X Jin, D Nguyen, W W Zhang, A P Kyritsis, and J A Roth Cell cycle arrest and inhibition of tumor cell proliferation by the p16INK4 gene mediated by an adenovirus vector Cancer Res. 1995 55: 3250-3253.

(8) Qiao J, Diaz R M, Vile R G Genome Biology 2004, 5(8):237 (29 Jul. 2004)

(9) Profiling of pathway-specific changes in gene expression following growth of human cancer cell lines transplanted into mice Creighton C, Kuick R, Misek D E, Rickman D S, Brichory F M, Rouillard J M, Omenn G S, Hanash S Genome Biology 2003, 4(7):R46 23 Jun. 2003

(10) Holt, Rinehart and Winston's Biology, Principles and Exploration, p 128

(11) Smeal T, Claus J, Kennedy B, Cole F, Guarente L: Loss of transcriptional silencing causes sterility in old mother cells of S. cerevisiae. Cell 1996, 84:633-642.

(12) Thomas H, Ougham H J, Wagstaff C, Stead A D: Defining senescence and death. J Exp Bot 2003, 54:1127-1132.

(13) Smeal T, Claus J, Kennedy B, Cole F, Guarente L: Loss of transcriptional silencing causes sterility in old mother cells of S. cerevisiae. Cell 1996, 84:633-642

(14) Boom R, Sol C J A, Jansen C L, Wertheim-van Dillen P M E & van der Noorda J (1990) Rapid and simple method for purification of nucleic acids. Journal of Clinical Microbiology, 28, 495-503.

(15) Matisoo-Smith E, Allen J S, Lagefoged T N, Roberts R M & Lambert D M (1997) Ancient DNA from Polynesian rats: extraction, amplification and sequence from single small bones. Electrophoresis, 18, 1534-1537.

(16) Onnnm R., Sol C., Beld M., Weel J., Goudsmit J., and Wertheim-van Dillen P. 1999. Improved Silica-Guanidiniumthiocyanate DNA Isolation Procedure Based on Selective Binding of Bovine Alpha-Casein to Silica Particles. Journal of Clinical Microbiology, March, Vol. 37, No. 3: 615-619

(17) (http://www-users.med.cornell.edu˜/jawagne/genes,_promoters,_DNA_&_ge.htmi

1. The use of Genome Replacement Therapy (GRT) in the creation of skin care products with genetic information of the patient within it to reduce the effects of wrinkles.
